(19) United States
(12) Patent Application Publication
(10) Pub. No.: US 2024/0374487 A1
(43) Pub. Date: Nov. 14, 2024

(54) ANTI-AGING AGENT, COSMETIC PRODUCT, WET TOWEL, AND HYGIENE PRODUCT

(71) Applicant: VB JAPAN TECHNOLOGY CO., LTD., Kunitachi-shi, Tokyo (JP)

(72) Inventors: Katsuaki DAN, Tokyo (JP); Katsuyuki FUJINAMI, Tokyo (JP)

(73) Assignee: VB JAPAN TECHNOLOGY CO., LTD., Kunitachi-shi, Tokyo (JP)

(21) Appl. No.: 18/691,949

(22) PCT Filed: Jun. 2, 2022
(86) PCT No.: PCT/JP2022/022408

§ 371 Date: Mar. 14, 2024

(30) Foreign Application Priority Data
Sep. 22, 2021 (JP) 2021-153913

(51) Int. Cl. A61K 8/19; A61K 8/02; A61Q 19/00; A61Q 19/08

(52) U.S. Cl. CPC A61K 8/19; A61K 8/0208; A61Q 19/007; A61Q 19/08

(57) ABSTRACT

An anti-aging agent that includes K₁₁H[(VO)₃(SbW₂O₃₃)₂] and/or Na₉[SbW₉O₃₃].

### TECHNICAL FIELD

The present invention relates to an anti-aging agent, a cosmetic product, a wet towel, and a hygiene product.

### BACKGROUND ART

While a glycation reaction, an oxidation reaction, and a chronic inflammatory reaction are pointed out as three major aging reactions of a living body, accumulation of reactive oxygen species (ROS) generated as a result of these reactions is known as a causative agent of various aging effects. The same applies to the skin cells, and it is considered that the suppression of the generation of wrinkles, stains, dullness, sagging and the like is achieved by the suppression of oxidation and glycation.

The glycation reaction is a non-enzymatic chemical reaction of amino acid residues in various proteins, and produces advanced glycation end products (AGE). AGEs play an important role in the aging of whole organisms and are involved in a wide variety of diseases. For example, AGE concentration in skin collagen increases with age and is higher in diabetic patients than in age-matched healthy individuals. Furthermore, the formation and accumulation of intracellular AGEs are known to be involved in skin aging, Alzheimer's disease, hypertension, arteriosclerosis and osteoporosis.

As the receptor of AGE, there are one that activates an intracellular signal pathway, particularly leads to inflammation, and is related to accumulation of ROS, and one that is related to degradation and removal of AGE. In order to prevent or delay cellular senescence, it is required to suppress the former function and promote the latter function. Examples of the former AGE receptor include RAGE and AGE-R2, and examples of the latter AGE receptor include FEEL-1, FEEL-2, AGE-R1, AGE-R3, and CD36.

The damage of DNA, protein, and lipid by ROS generated by hydrogen peroxide exposure or the like as oxidative stress plays an important role in the acceleration of aging and the onset and severity of geriatric diseases (For example, Non Patent Literatures 1 and 2). It is important to suppress accumulation of ROS and activate an antioxidant such as superoxide dismutase (SOD) in order to suppress aging.

It has been shown that, in skin moisturization and anti-aging, an increase in hyaluronic acid secretion associated with activation of fibroblasts, generation of Elastin, water molecule penetrating protein present on the cell membrane surface, aquaporin (AQP)-1 and -3 are involved, and it has also been revealed that AQP-1 and -3 are reduced by oxidative stress (for example, Non Patent Literature 3).

Elastin is a core protein of elastic fibers, is a cause of wrinkle formation, and is deeply involved in skin regeneration.

In recent years, heat shock protein (Hsp), which is a protein that is expressed when cells are stressed by heat, ultraviolet rays, active oxygen, and the like and protects cells, has attracted attention, and an expression inducer of heat shock protein has been proposed (see, for example, Patent Literature 1).

In addition, hundreds of polyacid compounds (polyoxometalates; PM compounds), which are a type of metal oxide and form a variety of structures, have been synthesized so far, and some having antitumor activity, antiviral activity, or antibacterial activity have been found. However, no knowledge has been obtained on the usefulness of the polyacid compound against ROS and the like and versatility of the polyacid compound against other substances.

### CITATION LIST

### Patent Literature

Patent Literature 1: WO 2012/043808 A1

### Non Patent Literature

Non Patent Literature 1: Kawanishi S, Hiraku Y, and Oikawa S. Mechanism of guanine-specific DNA damage by oxidative stress and its role in carcinogenesis and aging. Mutat Res, 488:65-76, 2001
Non Patent Literature 2: Saxena S, Vekaria H, Sullivan PG, and Seifert AW. Connective tissue fibroblasts from highly regenerative mammals are refractory to ROS-induced cellular senescence. Nat Commun. 27; 10 (1):4400, 2019
Non Patent Literature 3: Xu Y, Yao H, Wang Q, Xu W, Liu K, Zhang J, Zhao H, Hou G. Aquaporin-3 Attenuates Oxidative Stress-Induced Nucleus Pulposus Cell Apoptosis Through Regulating the P38 MAPK Pathway. Cell Physiol Biochem. 50 (5):1687-1697, 2018

### SUMMARY OF INVENTION

### Technical Problem

An object of the present invention is to provide an anti-aging agent using a polyacid compound and a cosmetic product containing the anti-aging agent, and to provide a wet towel and a hygiene product containing the cosmetic product.

### Solution to Problem

As a result of intensive studies, the present inventors have found that the above object is achieved by using a predetermined polyacid compound, and have completed the present invention.

That is, according to the present invention,
(1) an anti-aging agent containing K₁₁H[(VO)₃(SbW₉O₃₃)₂] and/or Na₉[SbW₉O₃₃],
(2) the anti-aging agent according to (1), which has an anti-glycation action,
(3) the anti-aging agent according to (2), which accelerates expression of at least one of FEEL-1, FEEL-2, CD36, AGE-R1, and AGE-R3,
(4) the anti-aging agent according to (2), which accelerates expression of at least one of Hsp104, gp96, Hsp90, Hsp70, Hsp60, and Hsp32;
(5) the anti-aging agent according to (1), which has an anti-oxidative stress action,
(6) the anti-aging agent according to (1), which has a skin moisturizing effect,
(7) the anti-aging agent according to (6), which has the skin moisturizing effect by at least one of expression of AQP-1, expression of AQP-3, secretion of hyaluronic acid, and generation of Elastin,
(8) a cosmetic product in which the anti-aging agent according to any one of (1) to (7) is blended;
(9) a wet towel containing the cosmetic product according to (8), and
(10) a hygiene product containing the cosmetic product according to (8)
are provided.

### Advantageous Effects of Invention

According to the present invention, it is possible to provide an anti-aging agent using a polyacid compound and a cosmetic product containing the anti-aging agent. Further, according to the present invention, it is possible to provide a wet towel and a hygiene product including the cosmetic product.

### BRIEF DESCRIPTION OF DRAWINGS

FIG. 1 is a graph showing expression of AGE receptor in a study of anti-glycation action in Examples.

FIG. 2 is a graph showing expression of heat shock protein in the study of anti-glycation action in Examples.

FIG. 3 is a graph showing the measurement results of ROS in the study of anti-glycation action in Examples.

FIG. 4 is a graph showing the measurement results of SOD in the study of anti-glycation action in Examples.

FIG. 5 is a graph showing the measurement results of cell viability in a study of anti-oxidation action in Examples.

FIG. 6 is a graph showing the measurement results of ROS in the study of anti-oxidation action in Examples.

FIG. 7 is a graph showing the measurement results of SOD in the study of anti-oxidation action in Examples.

FIG. 8 is a graph showing mRNA levels of AQP-1 in the study of anti-oxidation action in Examples.

FIG. 9 is a graph showing mRNA levels of AQP-3 in the study of anti-oxidation action in Examples.

FIG. 10 is a graph showing the production amount of hyaluronic acid in the study of anti-oxidation action in Examples.

FIG. 11 is a graph showing the production amount of Elastin in the study of anti-oxidation action in Examples.

### DESCRIPTION OF EMBODIMENTS

Hereinafter, an anti-aging agent of the present invention will be described. The anti-aging agent of the present invention contains K₁₁H[(VO)₃(SbW₉O₃₃)₂] and or Na₉[SbW₉O₃₃].

K₁₁H[(VO)₃(SbW₉O₃₃)₂] (Hereinafter, it may be referred to as VB2.) and Na₉[SbW₉O₃₃] (hereinafter, it may be referred to as VB3.) are compounds belonging to a metal oxide cluster called polyoxometalates (PM compound, polyacid compound). Here, the PM compound is a metal oxide cluster compound having a polyacid ion, and each compound belonging to the PM compound has unique biological activity such as antibacterial activity and antiviral activity as described in Journal of Materials Chemistry, Volume 15, Pages 4773-4782, 2005, Royal Society of Chemistry in the United Kingdom. The polyacid compound refers to a metal oxide cluster compound composed of a transition metal element (W(VI), V(V), etc.), and has a structure in which a tetrahedron or an octahedron in which an oxygen atom is usually coordinated to a metal atom or the like by 4 or 6 is used as a basic unit, and the basic unit is bonded via a ridge or a vertex.

Here, the polyacid compound used in the present invention is K₁₁H[(VO)₃(SbW₉O₃₃)₂] and Na₉[SbW₉O₃₃], and these may be used alone, or two kinds thereof formulated at a predetermined ratio may be used. The blending ratio when the two types are formulated at a predetermined ratio is not particularly limited, but Na₉[SbW₉O₃₃] is preferably 0.1 to 30 mol, and more preferably 10 to 20 mol, with respect to 1 mol of K₁₁H[(VO)₃(SbW₉O₃₃)₂]. In addition, it is particularly preferable to use 17.3 mol of Na₉[SbW₉O₃₃] with respect to 1 mol of K₁₁H[(VO)₃SbW₉O₃₃)₂].

In the present invention, VOSO₄ may be used as the metal oxide in addition to K₁₁H[(VO)₃SbW₉O₃₃)₂] and Na₉[SbW₉O₃₃]. VOSO₄ is a vanadium compound characterized by a diatomic ion VO²⁺. As the physiological activity, WO 99/17782 A1 and the like describe that it has a blood glucose lowering action. Here, when VOSO₄ is used, it is preferable to use 0.1 to 20 mol, and more preferable to use 4 to 8 mol of VOSO₄ with respect to 1 mol of K₁₁H[(VO)₃(SbW₉O₃₃)₂]. In addition, it is particularly preferable to use 5.5 mol of VOSO₄ with respect to 1 mol of K₁₁H[(VO)₃(SbW₉O₃₃)₂].

In addition, the use of the anti-aging agent of the present invention is not particularly limited, but for example, the anti-aging agent can be blended in cosmetic products. Examples of the cosmetic product include lotion, emulsion, facial cleanser, cleansing, serum, cream, foundation, eyebrow, mascara, eye shadow, eye liner, lipstick, gloss, blusher, white powder, and nail polish. In addition, as the form of cosmetic products, forms such as liquid, cream, solid, stick, and powder can be adopted.

The method for producing a cosmetic product is not particularly limited, but for example, a cosmetic product can be produced by mixing and stirring any component selected from cosmetic raw materials such as water, alcohols, oils, moisturizers, skin lightening agents, ultraviolet inhibitors, anti-wrinkle agents, peeling agents, fragrances, colorants, surfactants, chelating agents, antioxidants, thickeners, and pH adjusters in an aqueous solution of a polyacid.

Examples of the moisturizing agent include fulvic acid, hyaluronic acid, royal jelly, glycerin, and soybean extract. In addition, the fragrance is not particularly limited, and examples thereof include fragrance components having scents such as citrus, peppermint, lavender, Lindera, anise magnolia, Japanese cypress, Japanese cedar, and fir, and aromatic oils having these scents can be used.

The proportion of the anti-aging agent blended in the cosmetic product can be appropriately adjusted according to the intended use, but the proportion of the anti-aging agent blended in the cosmetic product is preferably 0.0001 to 80 wt %, more preferably 0.003 to 50 wt %, and still more preferably 0.005 to 30 wt %.

In addition, the above cosmetic products may be contained in known wet towel such as paper wet towel, nonwoven fabric wet towel, and cloth wet towel. As the cloth wet towel, for example, a towel can be used, and cotton or the like is used as the material of the towel. In addition, the cosmetic products may be contained in hygiene products such as hand soaps and hand sanitizers.

### EXAMPLES

Hereinafter, the present invention will be described with reference to Examples, but the present invention is not limited thereto. Note that parts and % in the present example are on a weight basis unless otherwise specified. In addition, the statistical significance of the measurement results was determined by t-test, and when P<0.05, it was determined that there was statistical significance in the difference between the two groups.

### Preparation of Polyacid Compound

Bulk powders of K₁₁H[(VO)₃(SbW₉O₃₃)₂] (hereinafter, it may be referred to as VB2.) and Na₉[SbW₉O₃₃] (hereinafter, it may be referred to as VB3.) were synthesized, respectively. The resulting bulk powder was ground in a mortar, dissolved in ultrapure water, and passed through a filter (pore size: 0.45 μm) to obtain aqueous solutions of VB2 and VB3, respectively. Here, the concentration of the obtained aqueous solution was 115 μg/mL for the VB2 aqueous solution and 1000 μg/mL for the VB3 aqueous solution.

### Reagent

DL-glyceraldehyde and hydrogen peroxide were purchased from Wako Pure Chemical Industries, Ltd., and bovine serum-derived albumin (fraction V) was purchased from Sigma-Aldrich.

### Preparation of AGE

Bovine serum-derived albumin (25 mg/mL) was dissolved in DL-glyceraldehyde, and cultured in 0.1 M of phosphate buffered saline (pH 7.4; hereinafter, it may be referred to as “PBS”.) at 37° C. for 1 week to obtain AGE.

### Cultivation of Cells

Normal human dermal fibroblasts (NHDF), juvenile foreskin (C-12300, PromoCell) were cultured in a dedicated medium, Fibroblast Growth Medium (C-23010, PromoCell) at 37° C. in a 5% CO₂ incubator.

### Examination of Anti-Glycation Action

### Measurement of Expression of AGE Receptor and Heat Shock Protein

First, as a co-existence group of VB and AGE, normal human dermal fibroblasts were treated with AGE (100 μg/mL), and the above VB2 (concentration after dilution: 115 μg/ml) and VB3 (concentration after dilution: 1000 μg/mL) diluted to an accurate 1/1000 amount were each added and retained at 37° C. for 4 hours.

In addition, as the VB single stimulation group, normal human dermal fibroblasts were not treated with AGE, and the above VB2 and VB3 diluted to an accurate 1/1000 amount were each added and held at 37° C. for 4 hours. Furthermore, as the AGE single stimulation group, normal human dermal fibroblasts were treated with AGE and kept at 37° C. for 4 hours without addition of VB2 or VB3. In addition, as an untreated group, normal human dermal fibroblasts were not treated with AGE, and were kept at 37° C. for 4 hours without adding VB2 or VB3.

Then, total RNA was extracted from the cells using Trizol reagent (Ambion), and mRNA levels of AGE receptor (FEEL-1, FEEL-2, CD-36, AGE-R1, AGE-R2, AGE-R3 and RAGE) and heat shock protein (Hsp104, gp96, Hsp90, Hsp70, Hsp60, and Hsp32) were measured by qRT-PCR (one-step quantitative reverse transcription-polymerase chain reaction) method.

The qRT-PCR method is specifically described in “qRT-PCR method” described later.

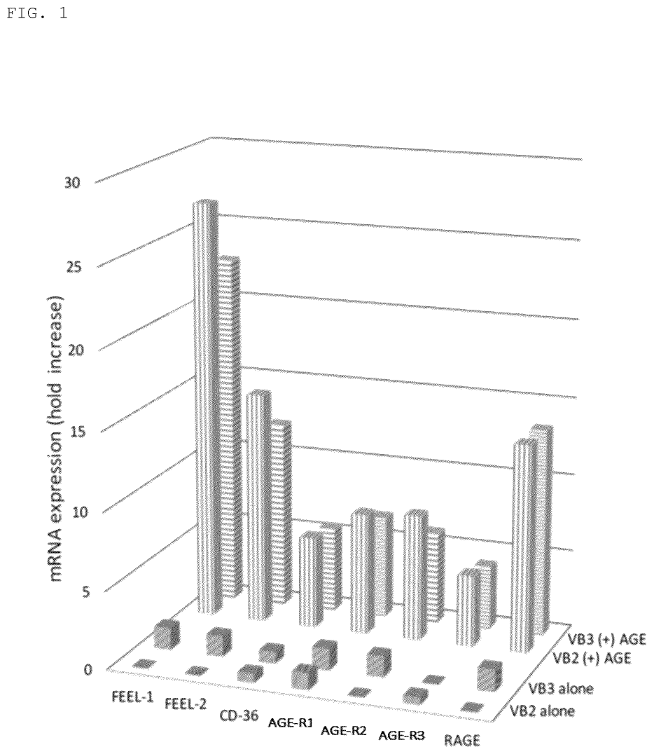

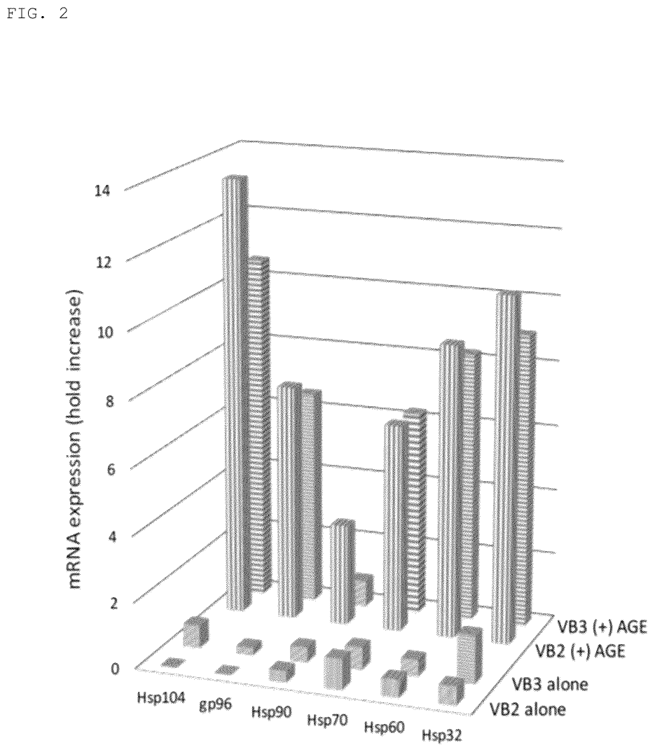

The measurement results for AGE receptor are shown in FIG. 1, and the measurement results for heat shock protein are shown in FIG. 2. In FIG. 1 and FIG. 2, for the co-existence group of AGE and VB, samples to which VB2 and VB3 were added are indicated as VB2(+)AGE and VB3(+)AGE, respectively, and these values were shown in comparison with the AGE single stimulation group. In addition, for the VB single stimulation group, samples to which VB2 and VB3 were added were indicated as VB2 alone and VB3 alone, respectively, and these values were shown in comparison with the untreated group.

As shown in FIG. 1, in the VB single stimulation group, there was no significant variation in the mRNA level of AGE receptor. On the other hand, in the co-existence group of AGE and VB, both VB2(+)AGE and VB3(+)AGE significantly increased the mRNA levels of FEEL-1, FEEL-2, and RAGE as compared with the mRNA level of AGE receptor in the AGE single stimulation group.

In addition, when VB2 and VB3 were each added to skin fibroblasts, the induction ability of mRNA of heat shock protein (hereinafter, it may be referred to as Hsp) as a stress response was examined, and as shown in FIG. 2, in the VB single stimulation group, the mRNA levels of various Hsps did not vary. On the other hand, in the co-existence group of AGE and VB, the mRNA levels of all Hsps other Hsp 90 among the examined Hsps were significantly increased as compared with the AGE single stimulation group.

### Measurement of Intracellular Reactive Oxygen (ROS) and Superoxide (SOD) Against AGE

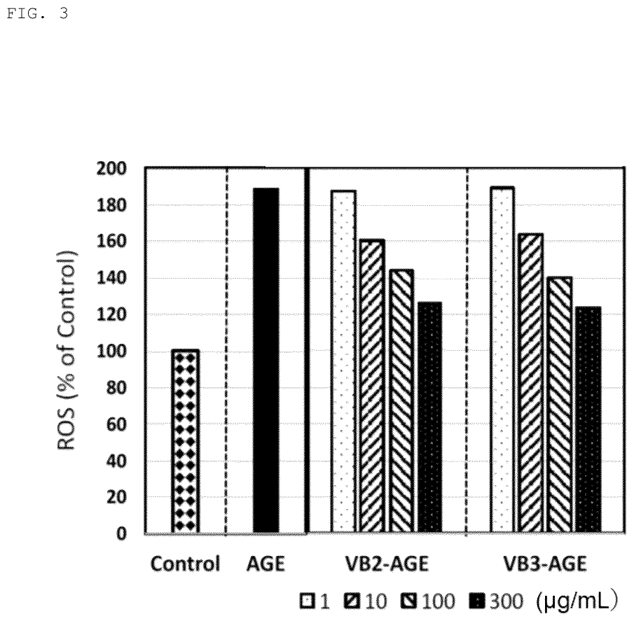

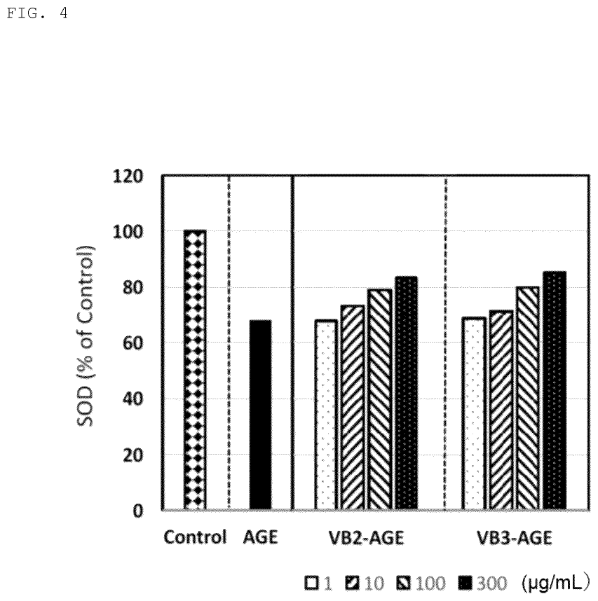

Normal human dermal fibroblasts were treated with AGE (100 μg/mL) and maintained at 37° C. for 4 hours with the addition of VB2 and VB3, respectively. Here, VB2 and VB3 were added at concentrations of 1 μg/mL, 10 μg/mL, 100 μg/mL, and 300 μg/mL, respectively. In FIGS. 3 and 4, which are graphs showing the results, those to which VB2was added were denoted as “VB2-AGE”, and those to which VB3 was added were denoted as “VB3-AGE”.

As Control, measurement was also performed on normal human dermal fibroblasts which were not treated with AGE and were held at 37° C. for 4 hours without addition of VB2 or VB3, and as an AGE single stimulation group, measurement was also performed on normal human dermal fibroblasts which were treated with AGE (100 μg/mL) and were held at 37° C. for 4 hours without addition of VB2 or VB3 (in FIGS. 3 and 4, it is denoted as “AGE”.).

Measurement of intracellular ROS was performed using fluorescence using dichlorofluorescin diacetate (DCF-DA; Invitrogen, Carlsbad, CA, USA). Specifically, cells were washed with PBS, and then incubated with 10 μM DCF-DA at 37° C. for 20 minutes. The intracellular ROS level was measured at an excitation wavelength of 525 nm using fluorescence intensity using the micro plate reader (SYNERGY/HT, BioTek, Japan). The results are shown in FIG. 3. FIG. 3 shows the results of each sample when Control is 100%.

Superoxide (SOD) was measured using SOD Assay Kit (Cayman Chemical, Ann arbor, MI, USA). Cells that had been subjected to various treatments were incubated for 24 hours, and then a Lysis buffer was added, homogenized, and centrifuged. Then, 10 μL of each sample was mixed with 200 μL of xanthine oxidase at room temperature. After 30 minutes, the absorbance at 450 nm was measured. The results are shown in FIG. 4.

FIG. 4 shows the results of each sample when Control is 100%.

### Examination of Anti-Oxidation Action

### Measurement of Cell Viability

Normal human dermal fibroblasts were cultured for 2 hours in a culture solution to which 0.2 mM hydrogen peroxide was added, then washed off with PBS, and replaced with a normal medium to continue the cultivation. VB2 and VB3 were each added at any timing before or after hydrogen peroxide was added, and co-cultured for 4 hours. Thereafter, the cells or the culture solution were recovered.

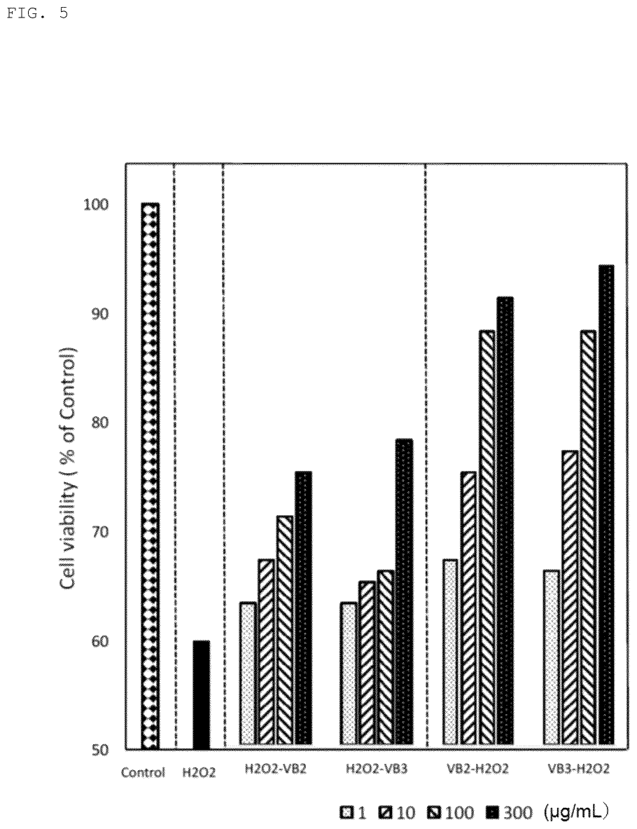

In FIG. 5 which is a graph showing the results, a sample obtained by adding VB2 or VB3 after adding hydrogen peroxide was denoted as “H2O2-VB2” and “H2O2-VB3”, respectively, and a sample obtained by adding hydrogen peroxide after adding VB2 or VB3 was denoted as “VB2-H2O2” and “VB3-H2O2”, respectively.

Here, VB2 and VB3 were added at concentrations of 1 μg/mL, 10 μg/mL, 100 μg/mL, and 300 μg/mL, respectively.

The normal human dermal fibroblasts to which neither hydrogen peroxide nor VB2 nor VB3 was added were treated in the same manner as described above, and measured as Control. In addition, a sample to which VB2 or VB3 was not added (in FIG. 5, it is denoted as “H2O2”) was also treated in the same manner as described above, and measurement was performed.

The number of surviving cells in normal human dermal fibroblasts was measured using Cell counting −8 kit (DOJINDO: , Japan). Control was displayed as a survival ratio (% of Control) by colorimetry with cells (450 nm). The results are shown in FIG. 5.

As shown in FIG. 5, the cell viability when normal human dermal fibroblasts were subjected to oxidative stress with 0.2 mM hydrogen peroxide was 59%. The cell viability upon addition of VB2 or VB3 following similar oxidative stress increased in a concentration-dependent manner, with maximum viability being 76% of 300 μg/mL for VB2, 78% of 300 μg/mL for VB3. On the other hand, when cells to which VB2 or VB3 had been added in advance were given similar oxidative stress later, the cell viability showed a significant increase in a concentration-dependent manner. Specifically, as compared with the sample to which VB2 or VB3 was added after the application of the oxidative stress, when VB2 or VB3 was added before the application of the oxidative stress, the increase was such that a significant difference was observed at 10 μg/mL or more.

### Measurement of Intracellular Reactive Oxygen (ROS) Production and Superoxide (SOD) Against Hydrogen Peroxide

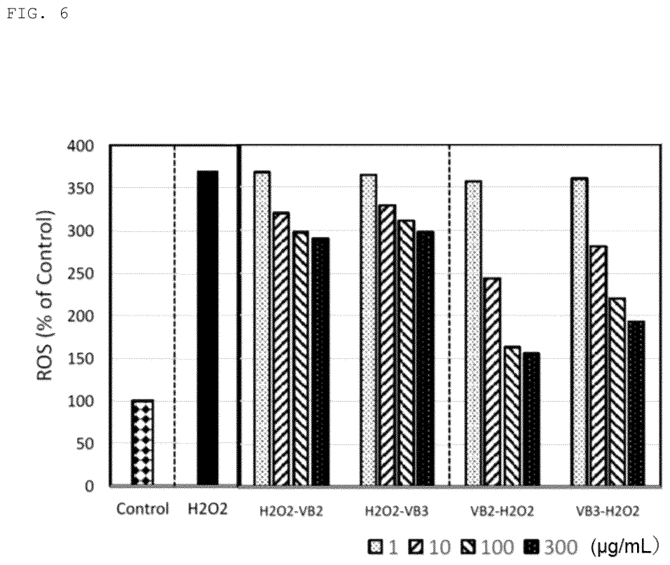

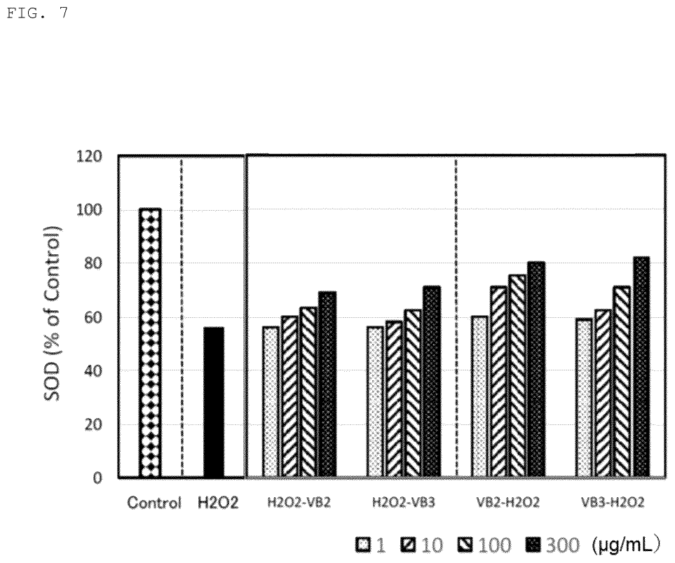

According to the procedure described in “Measurement of Cell Viability” above, normal human dermal fibroblasts were treated and cultured (co-culture), and cells or the culture solution was recovered. The concentrations of VB2 and VB3 used and the notation of the samples in FIGS. 6 and 7, which are graphs showing the results, are also similar to the above “Measurement of Cell Viability”.

The measurement of intracellular ROS and the measurement of superoxide (SOD) were performed by the same methods as the methods described in “Measurement of Intracellular Reactive Oxygen (ROS) Production and Superoxide (SOD) against AGE” above, respectively.

The measurement results of intracellular ROS and Superoxide (SOD) are shown in FIGS. 6 and 7, respectively. FIGS. 6 and 7 show the results of each sample when Control is 100%.

When the inhibitory effect on ROS generated by oxidative stress was examined, as shown in FIG. 6, the amount of intracellular fluorescence increased to 370% of that of normal cells by oxidative stress. When VB2 or VB3 was added before and after the oxidative stress, ROS production was suppressed in a concentration-dependent manner in both VB2 and VB3. In particular, when treatment with VB2 was performed in advance before application of oxidative stress, the production of ROS was further suppressed.

As shown in FIG. 7, when neither VB2 nor VB3 was added, the SOD amount was suppressed to 56% of Control by oxidative stress. When VB2 or VB3 was added before and after the oxidative stress, the SOD amount tended to be recovered in a concentration-dependent manner. In addition, the difference between the case where VB2 or VB3 was added before and after the application of oxidative stress, and the difference between the case where VB2 and VB3 were used were not clear.

### Study on Stimulatory Effect of Secretion of Aquaporin

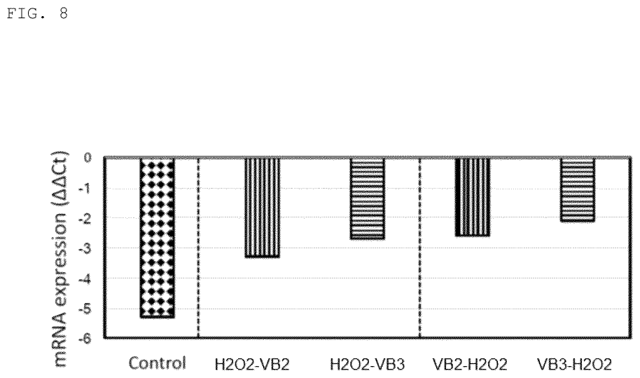

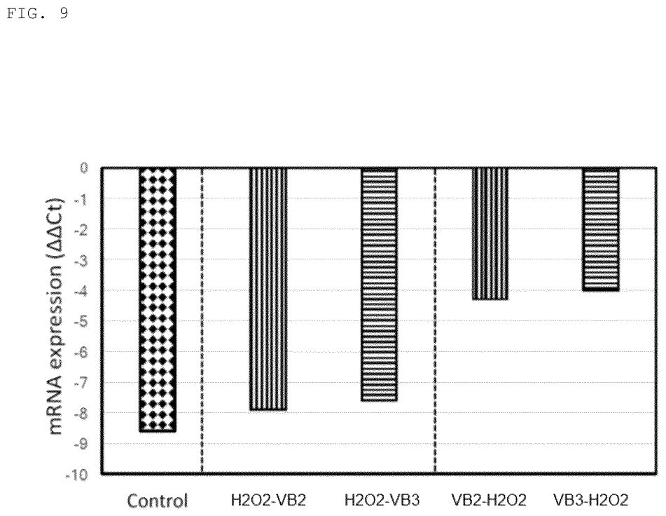

According to the procedure described in “Measurement of Cell Viability” above, normal human dermal fibroblasts were treated and cultured (co-culture), and cells or the culture solution was recovered. The notation of the sample in FIGS. 8 and 9, which are graphs showing the results, is also the same as in the “Measurement of Cell Viability” described above.

The measurement of aquaporin was performed for AQP-1 and AQP-3 by a qRT-PCR method, and the measurement result was obtained by a ΔΔCt method. FIG. 8 shows the results for AQP-1, and FIG. 9 shows the results for AQP-3. The qRT-PCR method is s specifically described in “qRT-PCR method” described later.

As a factor related to skin moisturization, when mRNA levels of AQP-1 and AQP-3 were measured by quantitative PCR, as shown in FIGS. 8 and 9, it was revealed that when oxidative stress is applied to normal human dermal fibroblasts, mRNA levels of AQP-1 and AQP-3 expressed on the cell surface decrease. For this decrease, a phenomenon in which the addition of VB2 or VB3, respectively, before or after the application of oxidative stress caused a repair, that is, a phenomenon in which the mRNA levels of AQP-1 and AQP-3 increased was observed. Its repair ability was observed with VB2 or VB3 regardless of the addition of VB2 or VB3 before or after the application of oxidative stress. Among them, the group to which VB2 or VB3 was added in advance before the application of oxidative stress tended to have a stronger repair ability than the group to which VB2 or VB3 was added after the application of oxidative stress. No significant difference was observed between VB2 and VB3.

### Production Amount of Hyaluronic Acid and Elastin Related to Skin Moisturizing Effect

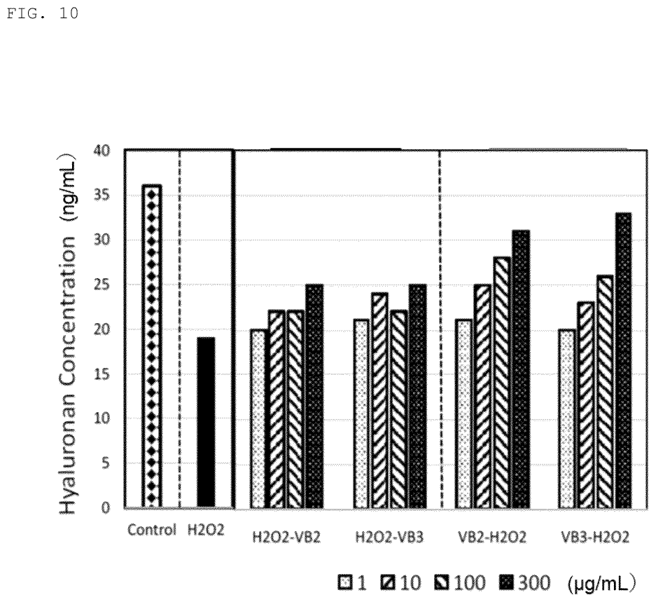

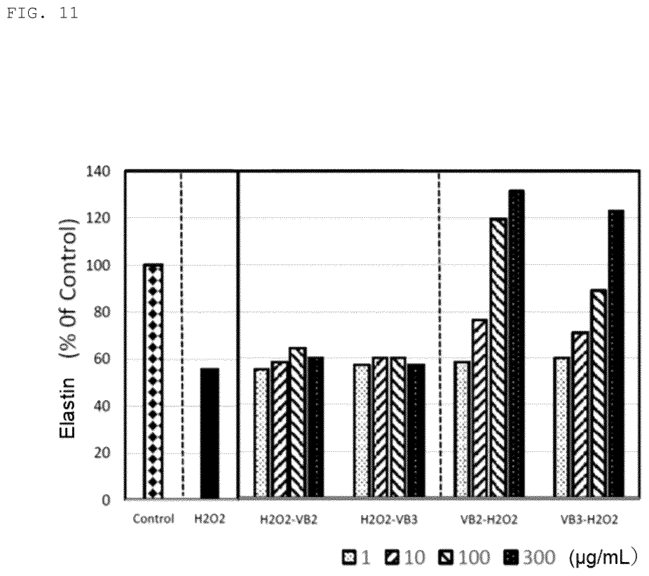

According to the procedure described in “Measurement of Cell Viability” above, normal human dermal fibroblasts were treated and cultured (co-culture), and cells or the culture solution was recovered. The concentrations of VB2 and VB3 used and the notation of the samples in FIGS. 10 and 11, which are graphs showing the results, are also similar to the above “Measurement of Cell Viability”.

The production amounts of hyaluronic acid and Elastin were measured by an ELISA method. The results of measuring the amount of hyaluronic acid secreted from normal human dermal fibroblasts into the culture supernatant by the ELISA method are shown in FIG. 10, and the results of measuring the amount of intracellular Elastin production by the ELISA method are shown in FIG. 11. FIG. 10 shows the results in terms of hyaluronic acid concentration, and FIG. 11 shows the results in terms of the ratio to the value of Control.

As shown in FIG. 10, the amount of hyaluronic acid was reduced to about half of Control by applying oxidative stress, but the amount of hyaluronic acid tended to be recovered by adding VB2 or VB3. Among them, the group to which VB2 or VB3 was added in advance before the application of oxidative stress tended to have a stronger repair ability than the group to which VB2 or VB3 was added after the application of oxidative stress. In the case of using VB2 and VB3, no significant difference was observed.

In addition, as shown in FIG. 11, the Elastin production amount was suppressed to 56% of Control by applying oxidative stress. Even when VB2 or VB3 was added after application of oxidative stress, the suppressed Elastin production amount could not be recovered. However, by adding VB2 or VB3 in advance before applying oxidative stress, a significant recovery of the amount of Elastin production was shown. In particular, at VB2, 100 μg/mL or more, and VB3, 300 μg/mL, the production amount was significantly higher than Control.

### qRT-PCR method

The qRT-PCR method used for the measurement of AGE receptor, heat shock protein, aquaporin, hyaluronic acid, and Elastin will be described. In the qRT-PCR method, 500 ng of the obtained total RNA was used as a template for PCR, and one step RT-PCR capable of performing CDNA synthesis by a reverse transcription reaction (Reverse Transcriptase; RT reaction) and quantitative PCR in one test tube was performed using Luna Universal One-Step qRT-PCR Kit.

As a PCR device, Thermal Cycler Dice Real Time System II manufactured by Takara was used. As one reaction system, 10 μL of Luna Universal One-Step Reaction Mix (2×), 1 μL of Luna WarmStart RT Enzyme Mix (20×), 0.8 μL of Forward primer (10 μM), 0.8 μL of Reverse primer, and Total RNA as a template for the PCR were used, and the total amount was adjusted to 20 μL in Nuclease-free Water.

The reaction was performed under the conditions of 1 cycle of 30 seconds at 95° C., and further cycles of 5 seconds at 95° C. and 30 seconds at 60° C. for 50 cycles. A variation in mRNA expression by 4 times or more (2 cycles as PCR reaction) was determined as a significant difference.

In the present invention, in order to pursue the versatility of two kinds of PM compounds (VB2 and VB3) reported to have antibacterial and antiviral activity, the reaction to AGE stimulation and several kinds of anti-oxidation actions on the application of oxidative stress using hydrogen peroxide were examined focusing on the anti-aging effect on the skin.

According to this, it was suggested that the addition of VB2 or VB3 alone to normal skin fibroblasts does not significantly change the cells, but the inhibitory action may be expressed when AGE stimulation or oxidative stress is loaded.

Normal human dermal fibroblasts are important cells that influence the state of the epidermis as well as keratinocytes of the epidermis while being present in the skin dermis.

As shown in FIGS. 1 and 2, VB2 and VB3 alone did not affect the mRNA expression of AGE receptor and Hsp in normal human dermal fibroblasts.

However, when AGE was added to normal human dermal fibroblasts as shown in FIG. 1, VB2 and VB3 increased the mRNA levels of AGE receptors, in particular FEEL-1, FEEL-2 and RAGE, almost in the same way. FEEL-1, FEEL-2 allow extracellular AGE to be taken up into cells by Endocytosis, and assist in degradation treatment with Lysosome. RAGE also increased, but a reaction to suppress the increase in ROS, which was verified in FIG. 3, was observed. This discrepancy can be explained by the assumption that the production of splice variants of RAGE is stimulated. That is, in addition to the full length RAGE (F-RAGE), it is known that two splicing variants of RAGE proteins are produced in cells. One is an intracellular domain-deleted type (C-terminally truncated RAGE: C-RAGE) of RAGE which is a soluble RAGE protein released from a cell membrane, and the other is an extracellular V-domain-deleted type (N-terminally truncated RAGE: N-RAGE).

In addition, Hsp that is expressed when cells are stressed by heat, ultraviolet rays, active oxygen, and the like and protects the cells was examined (FIG. 2).

Hsp104 exhibits a function of restoring aggregated proteins, gp96 is involved in intracellular antigen presentation, Hsp90 assists maturation of only selected proteins, and Hsp70 controls protein folding, transport, and degradation to control quality. In addition, Hsp60 is involved in folding maintenance of a protein in mitochondria and membrane permeation of a protein into mitochondria and the like, Hsp32 is involved in heme decomposition, and a heme decomposition product has an anti-oxidation action. Both VB2 and VB3 were shown to induce Hsp so as to retain the quality of intracellular proteins in cells subjected to AGE stress.

In addition, as shown in FIG. 5, in the experimental system in which the oxidative stress by hydrogen peroxide was applied such that the cell viability decreased by about 40%, VB2 or VB3 was added before and after the application of the oxidative stress, but the cell viability was higher and stronger cell protection effect was observed when VB2 or VB3 was added before the application of oxidative stress.

In addition, as shown in FIG. 6, although ROS in cells was increased by applying oxidative stress, the inhibitory effect was observed in a concentration-dependent manner for both VB2 and VB3, and the effect was also stronger when VB2 or VB3 was added before applying oxidative stress. In addition, as shown in FIG. 7, SOD, which is one of antioxidants, tended to recover the disappearance due to oxidative stress in conjunction with the above effects. However, it is considered that the disappearance of ROS cannot be explained only by the presence of an antioxidant such as SOD.

Fibroblasts are known to have a role of moisturizing the epidermis, and the presence of aquaporin, a receptor involved in the inflow of water molecules into cells, has attracted attention. In particular, AQP-1 and AQP-3 are expressed in fibroblasts. It has also been reported that hyaluronic acid secreted from cells is effective in moisturizing the skin. As shown in FIGS. 8 to 10, the mRNA expression of AQP-1 and AQP-3 and the secretion amount of hyaluronic acid were actually reduced by oxidative stress. When VB2 or VB3 was added thereto, the mRNA levels of AQP-1 and AQP-3 and the secretion amount of hyaluronic acid were improved. The improving action was stronger when VB2 or VB3 was present in advance before the application of oxidative stress.

In humans, half of the hyaluronic acid is present in the skin. While the hyaluronic acid decreases from the epidermis due to aging, the hyaluronic acid still remains in the dermis, which leads to a decrease in moisture, a decrease in elasticity, and atrophy of the skin due to aging. Use of VB2 or VB3 was shown to restore reduced hyaluronic acid.

In addition, it has been reported that the application of oxidative stress leads to a decrease in collagen and Elastin, and subsequently leads to the formation of wrinkles and a decrease in skin regeneration, and as shown in FIG. 11, the enhancing effect of Elastin is an effective effect on skin regeneration.

From the above, VB2 and VB3, which are polyacid compounds reported to have antimicrobial and antiviral activity, showed an anti-glycation and anti-oxidation action in a multifaceted manner against stress on skin fibroblasts caused by AGE and hydrogen peroxide, and in almost the same manner. Addition of VB2 or VB3 alone to the cells showed no variation in various parameters. From this, it was shown that both VB2 and VB3 are useful as anti-aging agents for skin.

1. An anti-aging agent comprising K₁₁H[(VO)₃(SbW₉O₃₃)₂] and/or Na₉[SbW₉O₃₃].

2. The anti-aging agent according to claim 1, wherein the anti-aging agent has an anti-glycation action.

3. The anti-aging agent according to claim 2, which accelerates expression of at least one of FEEL-1, FEEL-2, CD36, AGE-R1, and AGE-R3.

4. The anti-aging agent according to claim 2, which accelerates expression of at least one of Hsp104, gp96, Hsp90, Hsp70, Hsp60, and Hsp32.

5. The anti-aging agent according to claim 1, wherein the anti-aging agent has an anti-oxidative stress action.

6. The anti-aging agent according to claim 1, wherein the anti-aging agent has a skin moisturizing effect.

7. The anti-aging agent according to claim 6, wherein the anti-aging agent has the skin moisturizing effect by at least one of expression of AQP-1, expression of AQP-3, secretion of hyaluronic acid, and generation of Elastin.

8. A cosmetic product comprising the anti-aging agent according to claim 1 blended thereto.

9. A wet towel containing the cosmetic product according to claim 8.

10. A hygiene product containing the cosmetic product according to claim 8.
